(19) United States
(12) Patent Application Publication
(10) Pub. No.: US 2011/0117189 A1
(43) Pub. Date: May 19, 2011

(54) OPHTHALMIC COMPOSITIONS FOR TREATING PATHOLOGIES OF THE POSTERIOR SEGMENT OF THE EYE

(75) Inventors: Maria Grazia Mazzone, Acireale (IT); Claudine Civiale, Acicatena (IT); Francesco Cuffari, Catania (IT)

(73) Assignee: S.I.F.I. Societa' Industria Farmaceutica Italiana S.P.A., ACI S. Antonio (IT)

(21) Appl. No.: 13/003,143

(22) PCT Filed: Jul. 8, 2008
(86) PCT No.: PCT/IT08/00456

§ 371 Date: Jan. 7, 2011

(51) Int. Cl. A61K 9/127; A61K 31/573; A61K 9/14; A61P 27/02; A61P 31/00

(52) U.S. Cl. 424/450; 514/180; 424/489

(57) ABSTRACT

New compositions for ophthalmic use for the prevention and therapy of pathologies of the posterior segment of the eye. These compositions utilize xanthan gum as an active principle carrier, and can be advantageously administered as liquid-gel eye drops on the surface of the eye and optionally used in combination with other therapies for the treatment of the same pathologies.

### FIELD OF THE INVENTION

The present invention relates to the use of ophthalmic pharmaceutical compositions for the treatment and prevention of pathologies of the posterior segment of the eye. In particular, the invention relates to the ophthalmic use of compositions comprising xanthan gum as a carrier for active principles for the treatment and prevention of: uveitis, choroiditis, retinochoroiditis, chorioretinitis, retinal degeneration, age-related macular degeneration (AMD), retinal detachment, retinal neovascularisation, proliferative vitreoretinopathies, retinopathy of prematurity (ROP), posterior segment trauma, retinal vascular pathologies, endophthalmitis, macular edema, diabetic retinopathy, inflammatory pathologies of the retina, systemic pathologies with implications for the retina, possibly in combination with other therapies for the treatment of the same is pathologies.

### STATE OF THE ART

Pathologies of the posterior segment of the eye, and in particular retinal pathologies, are some of the more disabling pathologies of modern society. Numbered among these pathologies are those characterized by abnormal neovascularisation of the retina, iris and choroid (CNV), with consequent formation of dysfunctional neovessels which can cause leakage or haemorrhages, or can be associated with retinal edema, retinal/vitreous haemorrhage or retinal detachment resulting in the decline of visual acuity (Survey of Ophthalmology, January 2007, Vol. 52, S1, S3-S19). CNV is a degenerative pathology with multifactorial pathogenesis which comprises various components: pre-existing neovascularisation, further neovascularisation and inflammation. The ideal therapy should therefore act in a concerted manner on all these components. Unfortunately the therapies available today act on the individual components of CNV and are insufficient per se to overcome the therapeutic problem.

TABLE 1

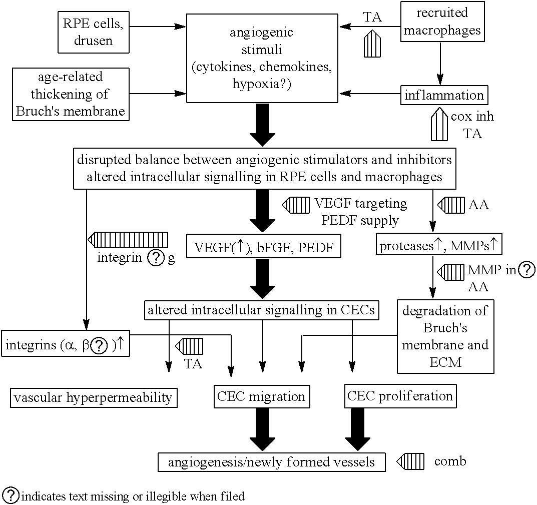

Angiogenic pathway (from Current Pharmaceutical Design, 2006, Vol. 12, 2645-2660)

Therapy for CNV has evolved rapidly in recent years. At first, (in the 1990's) thermal laser photocoagulation was used which is only applicable to a small number of patients (about 20%) since it cannot be carried out on central subfoveal CNVs and is also linked to reduced visual acuity due to the damage caused to photoreceptors adjacent to the irradiated region (Bradley, Review of Ophthalmology, 14 (10), 2007). Laser photocoagulation was then substituted by photodynamic therapy (PDT) specifically established to treat CNV in areas near the fovea without causing lesions in the surrounding irradiated tissue, and is currently constitutes the standard care in choroidal neovascularisation therapy. However, PDT only acts on neovessels already existing at the start of therapy, by selectively damaging their endothelial cytoskeleton resulting in their occlusion, thus stabilizing the neovascular lesion and slowing down—without however halting—visual acuity decline in patients affected by CNV (Kaiser, Retina Today, May/June 2007). At the present time, PDT is therefore considered to be an unsatisfactory therapy, as it is unable to improve the visual capability of the patient, but only to stabilize it (Augustin, Retina, The Journal of Retinal and Vitreous Diseases, 2007, Vol. 27, No. 2, 133-140).

Etiopathological studies on CNV have identified Vascular Endothelial Growth Factor (VEGF) as being among the main factors involved in angiogenesis associated with retinal pathologies (Eichler, Current Pharmaceutical Design, 2006, 12, 2645-2660; Bhisitkul, British Journal of Ophthalmology, 2006, 90, 1542-1547) they being also involved, together with angiopoietin, TGF-α, TGF-β and other growth factors, in the development of tumours (Ferrara, Laboratory Investigation, 2007, 87, 227-230). VEGF is actually up-regulated by the inflammatory process is underlying CNV, and has proangiogenic effects such as vasodilation, vascular permeability increase and proteolytic enzyme release with consequent tissue remodelling. These studies have led to research on the effect of anti-VEGF drugs (originally developed for oncological therapy) for treating CNV: hence pegaptanib (Macugen®, OSI Pharmaceuticals), bevacizumab (Avastin®, Genentech) and ranibizumab (Lucentis®, Genentech) have begun to be used in clinical therapy for CNV. Anti-VEGFs have a mechanism of action complementary to that of PDT in that they inhibit progression of neovascularisation, but do not act on pre-existing CNV: hence, for this reason, they are generally combined with PDT in clinical therapy.

Therapy with anti-VEGFs improves visual acuity in patients, but only if these drugs are administered frequently (i.e. monthly) for an extended time period (Lee, American Academy of Ophthalmology 2007 Annual Meeting, Scientific Paper PA 060 presented Nov. 12, 2007). Moreover, it appears that prolonged use of anti-VEGFs leads to a compensatory up-regulation of the receptors for said growth factor which can result in a rebound effect on cessation of the therapy (Augustin, Retina, The Journal of Retinal and Vitreous Diseases, 2007, Vol. 27, No. 2, 133-140).

Furthermore, currently available anti-VEGFs are administered by invasive means i.e. intravitreal injections, which are often associated with low patient compliance. Other drugs are currently under investigation for CNV treatment, namely: VEGF-Trap (which mimics the VEGF receptor and hence prevents its interaction with the real receptor), VEGF SiRNA (which block production of the mRNA specific for VEGF or its receptor), Tyrosine Kinase Inhibitors (TKIs, which function in a less specific manner, by blocking mediators of various growth factors, including VEGF), Vascular Disrupting Agents (VDAs, which bind specifically to neovessel tubulin causing their occlusion), substances which modulate the expression of endogenous antiangiogenic factors (such as angiostatin, endostatin, PEDF) and steroids and derivatives thereof (dexamethasone, triamcinolone acetonide, anecortave acetate) which act as angiostatics, by inhibiting the inflammatory component of the pathology and the up-regulation of VEGF supported thereby.

Each of the aforementioned product classes acts on a specific aspect of the is pathology (existing neovascularisation, further formation of neovessels or inflammatory component), and for this reason combinations of drugs with different mechanisms of action (PDT/steroids, PDT/anti-VEGFs) are now increasingly used in clinical practice to attack the pathology in a concerted manner and to reduce the treatments relative to the clinical protocol for monotherapy, while at the same time improving therapy safety and patent compliance (Piermarocchi, Paper presented at the International Congress of Ophthalmology “Fermo . . . AMD”, 14-15 Apr. 2005; Bradley, Angiogenesis, Vol. 10, No. 2, June 2007; Augustin, Ophthamology, January 2006, 113; Lee, American Academy of Ophthalmology 2007 Annual Meeting, Scientific Paper PA 060 presented Nov. 12, 2007; Augustin, Retina, The Journal of Retinal and Vitreous Diseases, 2007, Vol. 27, No. 2, 133-140).

TABLE 2
Antiangiogenic treatments and the relative clinical protocol

| Product | Clinical protocol |
|---|---|
| Ranibizumab | Every 30 days (intravitreal injection) |
| (Lucentis) | |
| Pegaptanib | Every 45 days (intravitreal injection) |
| (Macugen) | |
| Verteporfin | Every 90 days (intravenous injection + light: |
| (Visudyne) | 50 J/cm²) |

All the currently available therapies, such as PDT (intravenous injection of porphyrin derivatives and subsequent ocular irradiation) and treatment with corticosteroids or anti-VEGFs are carried out by invasive routes such as intravitreal injections or insertion of intraocular implants (Retisert®), because classical topical ophthalmic application does not enable effective concentrations of the active principle to be reached in the posterior chamber of the eye and particularly at the retina.

Moreover, these therapies do not resolve the pathology and the treatments must in any event be repeated over time.

Another negative aspect of the therapies so far described derives from the fact that they are often associated with the appearance of possibly serious side effects, related to the administration route, such as infectious endophthalmitis, retinal detachment and traumatic cataract (intravitreal injections, Eye, 2008, 1-2), clouding of the sight, subretinal/retinal haemorrhages, inflammation, photosensitivity reactions (PDT), necessitating removal of the bulbus due to serious side effects resulting from corticosteroid use in sustained-release intraocular devices.

In the light of the current knowledge the need was felt for new ophthalmic compositions which enable patients to be treated with non-invasive methods at low cost, such as classical topical administration, with the aim of also avoiding serious complications associated with invasive administration routes, but independent of the drug being administered (Eye, 2008, 1-2). Thus, applied research is being directed in this field without there being, for the moment, very positive signs.

TABLE 3

| | | | | | |
|---|---|---|---|---|---|
| | | | | | Expected |
| | | | | | date of |
| | Administration | Mechanism of | | Development | market |
| Molecule | route | action | Company | phase | launch |

Invasive administration route
Target: VEGF

| | | | | | |
|---|---|---|---|---|---|
| Bevasiranib | Intravitreal | VEGF SiRNA | OPKO | Phase III | 2010-2012 |
| VEGF Trap | Intravitreal | VEGF Trap | Regeneron | Phase III | 2010-2012 |
| | | | Bayer | | |
| AG 013958 | Subtenonian | VEGF TKI | Pfizer | Phase II | 2012-2014 |
| | injection | | | | |
| SiRNA 027 | Intravitreal | VEGF SiRNA | Allergan | Phase II | 2012-2014 |

Target: not VEGF

| | | | | | |
|---|---|---|---|---|---|
| Anecortave | Juxtascleral | Angiostatic cortisene | Alcon | Phase III | 2012-2014 |
| acetate | injection | | | | |
| AdGVPEDF | Intravitreal | PEDF gene therapy | Genvec | Phase I | 2014-2016 |
| Retinostat | Subretinal | Angiostatin/endostatin | Oxford | Start of clinical | >2016 |
| | injection | gene therapy | Biomedica | development: 2009 | |

Non-invasive administation route
Target: VEGF

| | | | | | |
|---|---|---|---|---|---|
| Vatalanib | Oral | VEGF TKI | Novartis | Phase II | 2012-2014 |
| TG100801 | Topical | VEGF TKI | Targegen | Phase II | 2012-2014 |
| | ophthalmic | | | | |
| Pazopanib | Topical | VEGF TKI | Glaxo | Phase II | 2012-2014 |
| | ophthalmic | | | | |

Target: not VEGF

| | | | | | |
|---|---|---|---|---|---|
| Combrestatin P | Topical | VDA | Oxigene | Start of clinical | >2016 |
| | ophthalmic | | | development:-second half | |
| | | | | of 2008 | |

### SUMMARY

New fluid ophthalmic compositions have now been identified, forming the subject of the present invention, which can enable a carried active principle to pass to the posterior chamber and in particular to the retina, following their topical application to the conjunctival sac. Said compositions are characterized by containing xanthan gum, an inexpensive sterilizable polymeric excipient able to give rise to transparent fluid compositions with a consistency such as to enable them to be administered as liquid gel eye-drops.

Furthermore, this polysaccharide polymer is compatible with various excipients used in ophthalmic pharmaceutics such as buffering agents, isotonizing agents, preservatives and other polymers, hence enabling pharmaceutical compositions to be obtained with characteristics suitable for topical ocular administration.

The compositions obtained using xanthan gum have a gel-like consistency and pseudoplastic rheological behaviour which gives them excellent compatibility with tears as they are completely miscible therewith and their viscosity diminishes during blinking.

The use of xanthan in ophthalmic compositions has been known since 1979 (U.S. Pat. No. 4,136,177, American Home Products Corporation) as a drug delivery system for ophthalmic compositions targeted to the anterior chamber of the eye, but not to the is posterior chamber or the retina. In U.S. Pat. No. 6,261,547 (Alcon) xanthan was also considered as an ophthalmic drug delivery system, but this patent considered compositions of aqueous solutions with total ionic strength of less than or equal to 120 mM which, following topical ophthalmic administration, were able to gel by interacting with lysozyme present in tears.

The new compositions of the invention are instead gel-like compositions, and are therefore fluid, having a total ionic strength greater than 120 mM, in particular of 150-170 mM, which can be administered to the eye as drops and do not change their physical state after administration. Said compositions have quite unpredictably enabled effective concentrations of active principles carried therein to reach the posterior chamber, and in particular the retina, after topical ophthalmic administration to the conjunctival sac.

Non-limiting examples of active principles carriable by means of the compositions of the invention include (from among all those administrable for treating pathologies of the posterior segment of the eye): anti-infectives (antibiotics, antibacterials, antivirals, antifungals), steroidal and non-steroidal anti-inflammatories, angiostatic cortisenes, COX inhibitors, antioxidants, angiogenesis inhibitors, neuroprotective agents, immunomodulating agents, vascular disrupting agents (VDA), immunosuppressant agents, antimetabolites, anti-VEGFs, associations and derivatives thereof.

The aforesaid active principles include as non-limiting examples: acyclovir, dexamethasone, desonide, betamethasone, triamcinolone, fluocinolone, fluorometholone, anecortave acetate, momethasone, fluoroquinolones, rimexolone, prednisolone, cephalosporin, tetracycline, anthracycline, chloramphenicol, aminoglycosides, sulfonamides, TNF inhibitors, anti-VEGF, anti-VEGF Mab, anti-PDGF, penicillins, macrolides, mycophenolate mofetil, methotrexate, thalidomide, lenalidomide, NOS inhibitors, COX-2 inhibitors, cyclosporine, cyclosporine A, Retinostat (Oxford Biomedica Plc), SiRNA-027 (Sirna Therapeutics Inc.), Cand5 (Acuity Pharmaceuticals), combrestatin (Oxigene), combrestatin-4-phosphate (Oxigene), MXAA (Novartis), AS1404 (Antisoma), 2-methoxyestradiol (Panzem, EntreMed), bevacizumab (Avastin, Genentech), ranibizumab (Lucentis, Genentech), pegaptanib sodium (Eyetech), ZD6126 (Angiogene), ZD6474 (Angiogene), growth factor antagonists, angiostatin (EntreMed), endostatin, anti TGF-α/β, anti IFN-α/β/γ, anti TNF-α, vasculostatin, vasostatin, angioarrestin and derivatives thereof.

The compositions of the invention can also comprise optional buffering agents, isotonizing agents and preservatives.

The ophthalmic compositions thus obtained show a surprising capacity for the active principle to penetrate to the posterior chamber of the eye and hence enable a targeted topical therapy to be undertaken, with high effectiveness and wide safety margin, suitable for preventing or treating pathologies in this ocular segment. The new therapy form can be used in combination with other known therapies for the same pathology.

By way of non-limiting example, pharmacokinetic data of compositions containing xanthan and dexamethasone sodium phosphate in the various ocular tissues, are given. Dexamethasone was chosen on the basis of its effectiveness and safety properties. Compared to triamcinolone acetonide, being often injected in suspension into the vitreous in combination with PDT, dexamethasone also acts on cell migration, causes fewer side effects on IOP and possesses antifibrotic and antiproliferative properties (Augustin, Retina, 27 (2): 133-140, 2007).

Dexamethasone is therefore characterized by a better therapeutic index than triamcinolone acetonide, but is currently administered, in AMD therapy, as a solution for intravitreal injection.

### DESCRIPTION OF THE FIGURES

FIG. 1: Distribution of dexamethasone in ocular tissues after topical ophthalmic single administration of a composition containing 0.15% dexamethasone sodium phosphate in a 1% xanthan base (overall data obtained from two pharmacokinetic experiments).

FIG. 2: Distribution of dexamethasone in the aqueous humour after topical ophthalmic single administration of 0.15% dexamethasone in a 1% xanthan base and of 0.15% dexamethasone in aqueous solution.

FIG. 3: Distribution of dexamethasone in the vitreous after topical ophthalmic single administration of 0.15% dexamethasone in a 1% xanthan base and of 0.15% dexamethasone in aqueous solution.

FIG. 4: Distribution of dexamethasone in the retina-choroid after topical ophthalmic single administration of 0.15% dexamethasone in a 1% xanthan base.

FIG. 5: Distribution of dexamethasone in plasma after topical ophthalmic single administration of 0.15% dexamethasone in a 1% xanthan base and of 0.15% dexamethasone in aqueous solution.

FIG. 6: Distribution of dexamethasone in plasma after topical ophthalmic single administration of 0.15% dexamethasone in a 1% xanthan base and of 0.15% dexamethasone in aqueous solution.

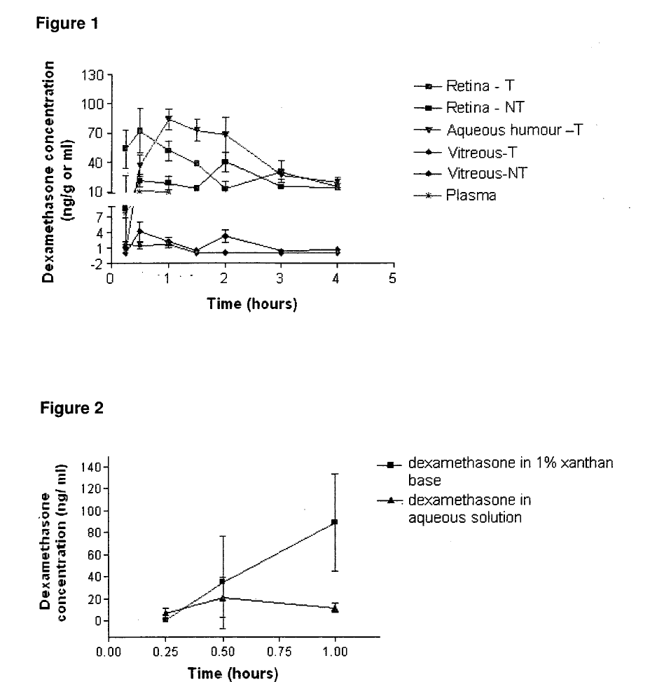

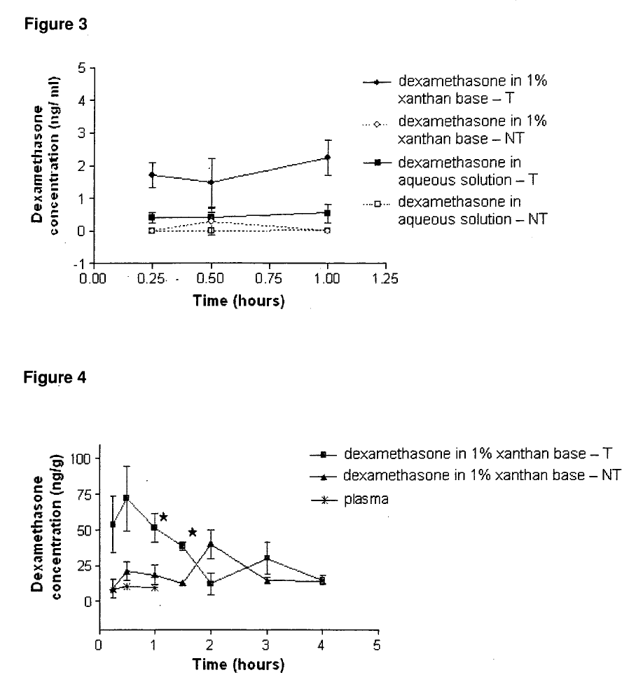

### DETAILED DESCRIPTION

The present invention relates to pharmaceutical compositions for ophthalmic use for treating pathologies of the posterior segment of the eye, comprising xanthan gum as a carrier for hydrophilic or lipophilic active principles, optionally encapsulated in suitable systems, such as: cyclodextrins, emulsions, microspheres, microcapsules, micro- and nano-particles, nanosystems, liposomes, lipospheres—as well as optional buffering agents, isotonizing agents and preservatives. Xanthan gum can be used at a concentration between 0.1 and 2% w/v, preferably between 0.2 and 1%. The compositions can be supplied to the patient in single-dose or multi-dose packs.

The buffering agent can be chosen from those known in the ophthalmic field, such as phosphate, phosphate-citrate, Tris, NaOH, histidine, tricine, lysine, glycine, serine, possibly adjusted to the correct pH with an acid component. The buffer is present in the composition at a concentration such that a pH between 5 and 8 is obtained/maintained, which is compatible with ocular tissue and with the carried active principle.

The isotonizing agent can be chosen from known ones, such as sodium chloride or citric acid, glycerol, sorbitol, mannitol, ethylene glycol, propylene glycol, dextrose and is present within a concentration range of, for example, from 0 to 1% w/v, rendering the composition isotonic with lacrimal fluid (270-310 mOsm/kg).

The aforesaid buffering and isotonizing agents, although useful and preferred, are not imperative for the purposes of the present invention.

The compositions of the invention formulated in multi-doses can also contain antimicrobial preservatives such as: parabens, quaternary ammonium salts, polyhexamethylene biguanidine (PHMB) and others from those usable in compositions for ophthalmic use. The solvent used in the compositions is preferably water or an aqueous solution of one or more components compatible with topical ophthalmic use.

The compositions of the invention can also contain other ionic polymers (such as hyaluronic acid) and non-ionic polymers (such as cellulose and its derivatives).

In its general meaning the process for preparing the compositions described herein comprises mixing, in a suitable solvent, the active principle and the polymer component. Said process forms a further aspect of the invention. The following preparation method is given by way of non-limiting example.

Two solutions are prepared of each component at double concentration (2×).

Xanthan gum is placed in one solution, and agitated until completely dissolved. Dexamethasone sodium phosphate and the salts are dissolved in the other. The solution containing xanthan is then sterilized in an autoclave. The solution containing dexamethasone sodium phosphate is instead sterilized by filtration. The two solutions are then stirred together under magnetic agitation in a sterile environment, until a single solution is obtained.

For the purposes of administration, the aforesaid compositions are preferably produced as liquid gel eye-drops for ophthalmic use, either in single-dose or multi-dose. These compositions can be produced by suitably varying the concentration of the polymer component (and hence composition viscosity) and/or adding additional components, such as other ionic or non-ionic polymers. Methods for producing said alternative forms are known in the art. The present compositions enable the active principle carried therein to penetrate specific parts of the eye, in particular the posterior chamber and the retina, following topical ophthalmic administration. They therefore enable safety and patient compliance to be improved by avoiding recourse to intravitreal injection or insertion of a depot into the vitreous or under the conjunctiva or at the retina. In contrast to intravitreal injections or insertion of medicated inserts, the compositions of the invention enable the therapy to be stopped immediately, as soon as undesired or toxic effects caused by the active principle are noted.

A further aspect of the invention is therefore the topical ophthalmic use of the compositions as aforedefined in preparing a medicament for the treatment or prevention of pathologies of the posterior chamber of the eye, and in particular the retina. The said compositions are also compatible with intravitreal or periocular administration. The invention also includes the use of the described compositions for the treatment and prevention of retinal pathologies, comprising the administration, to a patient requiring it, of a therapeutically effective quantity of the compositions as aforedefined. Conditions of the retina which can be effectively treated for the purposes of the invention include the following: uveitis, choroiditis, retinochoroiditis, chorioretinitis, retinal degeneration, age-related macular degeneration (AMD), retinal detachment, retinal neovascularisation, proliferative vitreoretinopathy, retinopathy of prematurity (ROP), posterior segment trauma, retinal vascular pathologies, endophthalmitis, macular edema, diabetic retinopathy, inflammatory pathologies of the retina, systemic pathologies with implications for the retina, possibly in combination with other therapies for the treatment of the same pathologies.

It has surprisingly been observed that the active principle in the compositions of the invention, administered to the conjunctival sac by topical ophthalmic means, is able to reach the retina-choroid at therapeutically effective concentrations.

The advantages relating to the higher bioavailability of the carried active principle in the compositions of the invention have been achieved without requiring recourse to additional substances such as cyclodextrins or penetration enhancers which would render preparation of the final composition more expensive. The following examples further illustrate the invention without limiting it.

### EXPERIMENTAL PART

### Example 1

### Liquid Gel in a 1% Xanthan Base not Containing Antimicrobial Preservative

### 1.1 Composition

| Components | % w/v |
|---|---|
| Xanthan | 1.0000 |
| Dexamethasone sodium phosphate | 0.1500 |
| Disodium phosphate dodecahydrate | 0.5000 |
| Sodium phosphate monobasic monohydrate | 0.1465 |
| Sodium citrate dihydrate | 2.1000 |
| Purified water | q.s. to 100 ml |

### 1.2 Composition

| Components | % w/v |
|---|---|
| Xanthan gum | 1.0000 |
| Sodium chloride | 0.1500 |
| Potassium chloride | 0.1500 |
| Magnesium chloride hexahydrate | 0.0120 |
| Calcium chloride dihydrate | 0.0084 |
| Glycerol | 0.5000 |
| Desonide sodium phosphate | 0.2500 |
| Sodium citrate dihydrate | 0.0590 |
| Disodium phosphate dodecahydrate | 0.4100 |
| Sodium phosphate monobasic monohydrate | 0.1600 |
| Purified water | q.s. to 100 ml |

### 1.3 Composition

| Components | % w/v |
|---|---|
| Xanthan gum | 1.0000 |
| Netilmycin sulphate | 0.4550 |
| Dexamethasone sodium phosphate | 0.1320 |
| Sodium citrate dihydrate | 2.1000 |
| Disodium phosphate dodecahydrate | 0.5000 |
| Sodium phosphate monobasic monohydrate | 0.1465 |
| Purified water | q.s. to 100 ml |

### Example 2

### Liquid Gel in a 0.2% Xanthan Base not Containing Antimicrobial Preservative

### 2.1 Composition

| Components | % w/v |
|---|---|
| Xanthan gum | 0.2000 |
| Dexamethasone sodium phosphate | 0.1500 |
| Tris base | 0.2423 |
| Sodium chloride | 0.1500 |
| Potassium chloride | 0.1500 |
| Sodium citrate dihydrate | 0.0590 |
| Magnesium chloride hexahydrate | 0.0120 |
| Calcium chloride dihydrate | 0.0084 |
| Glycerol | 0.5000 |
| 1M HCl | q.s. to pH 7.4-7.5 |
| Purified water | q.s. to 100 ml |

### 2.2 Composition

| Components | % w/v |
|---|---|
| Xanthan gum | 0.2000 |
| Dexamethasone sodium phosphate | 0.1500 |
| Sodium citrate dihydrate | 0.0590 |
| Sodium chloride | 0.1500 |
| Potassium chloride | 0.1500 |
| Magnesium chloride hexahydrate | 0.0120 |
| Calcium chloride dihydrate | 0.0084 |
| Glycerol | 0.8400 |
| 1M HCl | q.s. to pH 7.4-7.5 |
| Purified water | q.s. to 100 ml |
| Tris base | 0.2423 |

### Example 3

### Liquid Gel in a 1% Xanthan Base Containing Antimicrobial Preservative

### 3.1 Composition

| Components | % w/v |
|---|---|
| Xanthan gum | 1.0000 |
| Sodium chloride | 0.1500 |
| Potassium chloride | 0.1500 |
| Glycerol | 0.5000 |
| Dexamethasone sodium phosphate | 0.1500 |
| Sodium citrate dihydrate | 0.0590 |
| Tris base | 0.2423 |
| Benzalkonium chloride | 0.0050 |
| Edetate disodium | 0.1000 |
| 1M HCl | q.s. to pH 7.4-7.5 |
| Purified water | q.s. to 100 ml |

### 3.2 Composition

| Components | % w/v |
|---|---|
| Xanthan gum | 1.0000 |
| Desonide sodium phosphate | 0.2500 |
| Disodium phosphate dodecahydrate | 0.4100 |
| Sodium phosphate monobasic monohydrate | 0.1600 |
| Potassium chloride | 0.1500 |
| Sodium citrate dihydrate | 0.0590 |
| Edetate disodium | 0.0100 |
| Glycerol | 0.5000 |
| Benzalkonium chloride | 0.0050 |
| Sodium chloride | 0.1500 |
| Purified water | q.s. to 100 ml |

### 3.3 Composition

| Components | % w/v |
|---|---|
| Xanthan gum | 1.0000 |
| Dexamethasone sodium phosphate | 0.1500 |
| Tris base | 0.2420 |
| Sodium citrate dihydrate | 0.0590 |
| Sodium chloride | 0.1500 |
| Potassium chloride | 0.1500 |
| Magnesium chloride hexahydrate | 0.0120 |
| Calcium chloride dihydrate | 0.0084 |
| Glycerol | 0.5000 |
| Sodium perborate hydrate | 0.0300 |
| 1M HCl | q.s. to pH 7.4-7.5 |
| Purified water | q.s. to 100 ml |

### 3.4 Composition

| Components | % w/v |
|---|---|
| Xanthan gum | 1.0000 |
| Hydroxyethyl cellulose | 0.4000 |
| Netilmycin sulphate | 0.4550 |
| Dexamethasone sodium phosphate | 0.1320 |
| Sodium citrate dihydrate | 2.1000 |
| Disodium phosphate dodecahydrate | 0.7000 |
| Sodium phosphate monobasic monohydrate | 0.0680 |
| Benzalkonium chloride | 0.0050 |
| Purified water | q.s. to 100 ml |

### Example 4

### Liquid Gel in a 0.2% Xanthan Base Containing Antimicrobial Preservative

### 4.1 Composition

| Components | % w/v |
|---|---|
| Xanthan gum | 0.2000 |
| Dexamethasone sodium phosphate | 0.1500 |
| Disodium phosphate dodecahydrate | 0.0890 |
| Sodium phosphate monobasic monohydrate | 0.0350 |
| Sodium chloride | 0.1500 |
| Potassium chloride | 0.1500 |
| Sodium citrate dihydrate | 0.0590 |
| Magnesium chloride hexahydrate | 0.0120 |
| Calcium chloride dihydrate | 0.0084 |
| Glycerol | 0.8400 |
| Benzalkonium chloride | 0.0050 |
| Purified water | q.s. to 100 ml |

### Example 5

### In-Vivo Pharmacokinetic Tests and Comparison with an Aqueous Solution of the Same Active Principle Concentration

### 5.1 Methodology

Ocular distribution of dexamethasone was determined in pigmented rabbits after single administration to the conjunctival sac of a composition containing 1% xanthan and 0.15% dexamethasone sodium phosphate, and compared with an aqueous solution containing the same concentration of active principle. Dexamethasone concentration in ocular tissues was determined by a LC/MS/MS method.

### Materials and Methods

Two experiments were carried out on male pigmented rabbits weighing 1.8-2.2 kg divided into 2 treatment groups, of which only one eye was treated with: 50 μl of the composition containing dexamethasone sodium phosphate in 1% xanthan base (Group I) or 50 μl of dexamethasone sodium phosphate in solution (Group II). In the first experiment the animals were then killed at the following times: 30, 60, 90, 120, 180 and 240 minutes after treatment (n=6 animals for each time point). In the second experiment the animals were killed at these times: 15, 30 and 60 minutes after treatment (n=4 animals for each time point). The active principle was determined in the aqueous humour, vitreous, retina-choroid and plasma after suitable extraction from the biological tissue, by a LC/MS/MS method.

### Results

The results obtained from analysis of the data from the two experiments for the treated and non-treated eyes of Group I animals (treatment: dexamethasone sodium phosphate in 1% xanthan base), shown in tables 1 and 2, were expressed graphically to compare the quantitative data acquired for the treated eye (T) with data relating to the contralateral non-treated eye (NT, FIG. 4).

TABLE 1
Group I, treated eye

| | | Cₘₐₓ | AUCₐₗₗ |
|---|---|---|---|
| | Tₘₐₓ (h) | (ng/g or ml) ± SE | (h*ng/g or ml) ± SE |
| Aqueous | 1 | 83.72 ± 10.64 | 178.92 ± 17.08 |
| humour | | | |
| Vitreous | 1 | 1.76 ± 0.59 | 1.88 ± 0.38 |
| Retina-choroid | 0.5 | 67.79 ± 22.73 | 129.92 ± 16.81 |

TABLE 2
Group I, non-treated eye

| | | Cₘₐₓ | AUCₐₗₗ |
|---|---|---|---|
| | Tₘₐₓ (h) | (ng/g or ml) ± SE | (h*ng/g or ml) ± SE |
| Aqueous | — | — | — |
| humour | | | |
| Vitreous | 0.5 | 4.24 ± 1.89 | 6.19 ± 1.25 |
| Retina-choroid | 2 | 39.98 ± 9.97 | 77.14 ± 9.02 |

In particular, as seen from the results, the AUCs for the retina-choroid tissue for the treatment groups are significantly different (p<0.05, test: Student's T).

### Aqueous Humour

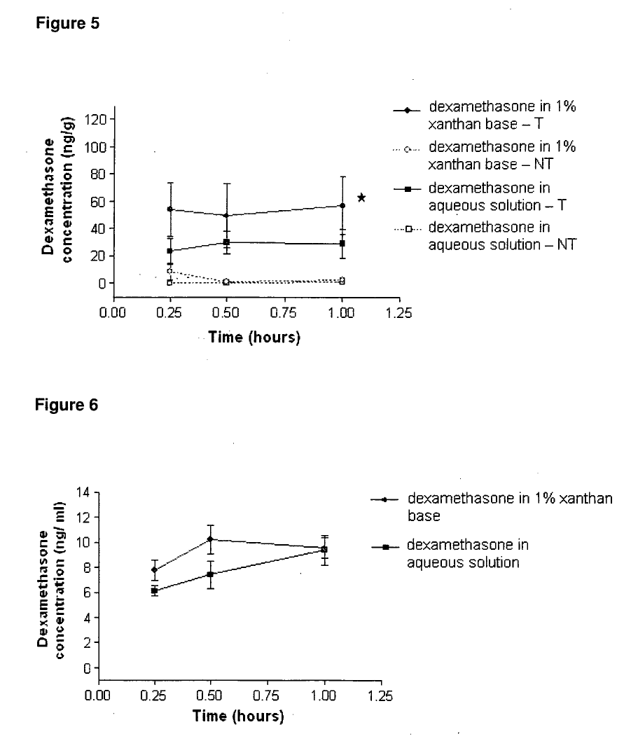

Concentrations of dexamethasone found in the aqueous humour of eyes treated with the composition of dexamethasone sodium phosphate in 1% xanthan base were significantly higher than those of the aqueous humour samples derived from non-treated eyes (Tables 1-2, FIG. 5), where virtually negligible concentrations of active principle were found (about 1 ng/ml). These values were also compared with those derived from analysis of the aqueous humour samples derived from is eyes treated with aqueous dexamethasone solution. As deduced from FIG. 5, dexamethasone concentration in the aqueous humour in the first hour following the single ocular treatment undertaken with 0.15% dexamethasone in 1% xanthan base was significantly higher than the concentration found, in the same tissue, after single administration of aqueous dexamethasone solution at the same zo concentration.

### Vitreous

Concentrations of dexamethasone found in the vitreous of treated and non-treated eyes were less than 10 ng/ml (Tables 1-2, FIG. 6).

### Retina-Choroid

Pharmacokinetic analysis of retina-choroid tissue after ocular single administration of 0.15% dexamethasone in 1% xanthan base has shown high concentrations of the active principle both in the treated eyes and the contralateral non-treated eyes, but in tissue derived from treated eyes, higher concentrations of dexamethasone are reached, especially in the first 90 minutes after treatment. The AUC relating to dexamethasone concentrations in treated eyes is significantly greater than the AUC for non-treated eyes. Furthermore, dexamethasone concentrations in the retina-choroid are higher than concentrations measured in plasma (FIGS. 7 and 9). By comparing data on dexamethasone concentrations in the retina-choroid after treatment with the xanthan-containing composition and with the aqueous solution, it can be seen that in addition to the systemic passage of the active principle, which is comparable for both formulations (FIG. 8), in this tissue absorption is greater for the compositions containing xanthan through the conjunctiva and sclera.

In conclusion, the experimental data show that, in the treated eye, the xanthan-containing dexamethasone composition results, for the same treatment (single administration), in a higher concentration of active principle in the aqueous humour and retina-choroid than does the aqueous solution for the same active principle concentration. The presence of the active principle was also observed in the retina-choroid of the non-treated contralateral eye. This clearly shows a systemic passage which however by itself does not justify the concentrations of dexamethasone found in the tissues of the posterior chamber of treated eyes, after treatment with the xanthan-containing composition.

The presence of xanthan was found to increase topical bioavailability of dexamethasone in the retina-choroid in a manner sufficient to perform the required anti-inflammatory and antiangiogenic action.

1-30. (canceled)

31. A pharmaceutical composition for therapeutic use comprising xanthan gum as carrier of a therapeutically effective amount, sufficient for the treatment or prevention of pathologies of the posterior segment of the eye and in particular the retina, of an active principle selected from the group consisting of anti-infectives (antibiotics, antibacterials, antivirals, antifungals), steroidal and non-steroidal antiinflammatories, angiostatic cortisenes, COX inhibitors, antioxidants, angiogenesis inhibitors, neuroprotective agents, immunomodulating agents, vascular disrupting agents (VDA), immunosuppressant agents, antimetabolites and anti-VEGF.

32. Pharmaceutical composition according to claim 31, wherein the active principle is acyclovir, dexamethasone, desonide, betamethasone, triamcinolone, fluocinolone, fluorometholone, anecortave acetate, momethasone, fluoroquinolones, rimexolone, prednisolone, cephalosporin, tetracycline, anthracycline, chloramphenicol, aminoglycosides, sulfonamides, TNF inhibitors, anti-VEGF, anti-VEGF Mab, anti-PDGF, penicillins, macrolides, mycophenolate mofetil, methotrexate, thalidomide, lenalidomide, NOS inhibitors, COX-2 inhibitors, cyclosporine, cyclosporine A, SiRNA-027, Candy, combrestatin, combrestatin-4-phosphate, MXAA, AS1404, 2-methoxyestradiol, bevacizumab, ranibizumab, pegaptanib sodium, ZD6126, ZD6474, growth factor antagonists, angiostatin, endostatin, anti TGF-α/β, anti IFN-α/β/γ, anti TNF-α, vasculostatin, vasostatin, angioarrestin and derivatives or mixtures thereof.

33. Pharmaceutical composition according to claim 31 wherein the active principle is incorporated as such or in a suitable delivery system such as cyclodextrins, emulsions, microspheres, microcapsules, microparticles, nanoparticles, nanosystems, liposomes, lipospheres.

34. Pharmaceutical composition according claim 31 comprising xanthan gum in a quantity between 0.1 and 2%.

35. Pharmaceutical composition according to claim 34 comprising xanthan gum in a quantity between 0.2 and 1%.

36. Pharmaceutical composition according to claim 31 comprising anionic or neutral polymers as the excipients.

37. Pharmaceutical composition according to claim 36 wherein the anionic polymer is hyaluronic acid.

38. Pharmaceutical composition according to claim 36 wherein the neutral polymer is cellulose or a derivative thereof.

39. Pharmaceutical composition according to claim 31 in the form of a liquid gel with a total ionic strength greater than 120 mM.

40. Pharmaceutical composition according to claim 39 with a total ionic strength equal to 150-170 mM.

41. Pharmaceutical composition according to claim 31 with a pH between 5 and 8, compatible with ocular tissues and the carried active principles.

42. Pharmaceutical composition according to claim 41 wherein the pH value is achieved by means of suitable buffering agents for ophthalmic use.

43. Pharmaceutical composition according to claim 31 being isotonic with lacrimal fluid (270-310 mOsm/kg).

44. Pharmaceutical composition according to claim 43 wherein isotonicity is obtained by means of isotonizing agents suitable for ophthalmic use.

45. Pharmaceutical composition according to claim 31 further containing antimicrobial preserving agents.

46. Pharmaceutical composition according to claim 31 in form of an aqueous solution.

47. Pharmaceutical composition according to claim 31 for topical administration onto the surface of the eye for the treatment or prevention of pathologies of the posterior chamber of the eye and in particular the retina.

48. Pharmaceutical composition according to claim 47 wherein the pathologies are selected from the group consisting of choroiditis, retinochoroiditis, chorioretinitis, retinal degeneration, retinal neovascularisation, age-related macular degeneration (AMD), retinal detachment, proliferative vitreoretinopathy, retinopathy of prematurity (ROP), posterior segment trauma, inflammatory pathologies of the retina and systemic pathologies with implications for the retina.

49. Process for preparing a composition according to claim 31 wherein two previously sterilized solutions containing respectively the active principle or active principles with optional excipients and the xanthan gum, are mixed under aseptic conditions.
